(19) United States
(12) Patent Application Publication
(10) Pub. No.: US 2020/0315514 A1
(43) Pub. Date: Oct. 8, 2020

(54) DIGITAL BIOMARKERS FOR MUSCULAR DISABILITIES

(71) Applicant: Hoffmann-La Roche Inc., Little Falls, NJ (US)

(72) Inventors: Christian Gossens, Basel (CH); Michael Lindemann, Basel (CH)

(21) Appl. No.: 16/904,343

(22) Filed: Jun. 17, 2020

Related U.S. Application Data
(63) Continuation of application No. PCT/EP2018/086192, filed on Dec. 20, 2018.

(30) Foreign Application Priority Data
Dec. 21, 2017 (EP) 17 209 699.2

(51) Int. Cl. A61B 5/22; G16H 70/60; G16H 50/30; G16H 50/20; G16H 50/50; G16H 40/67; A61B 5/00; A61B 5/11; G06F 3/041; G01L 1/14

(52) U.S. Cl. CPC A61B 5/225; A61B 2562/0247; G16H 50/30; G16H 50/20; G16H 50/50; G16H 40/67; A61B 5/4082; A61B 5/6898; A61B 5/681; A61B 5/7282; A61B 5/7435; A61B 5/7475; A61B 5/1124; G06F 3/0414; G01L 1/142; A61B 5/1101; G16H 70/60

(57) ABSTRACT

A method of assessing a muscular disability and, preferably, spinal muscular atrophy (SMA) in a subject is disclosed. A performance parameter is determined from a dataset of pressure measurements of the individual finger strength from the subject using a mobile device and is compared to a reference whereby the muscular disability and, preferably, SMA is assessed.

### RELATED APPLICATIONS

This application is a continuation of PCT/EP2018/086192, filed Dec. 20, 2018, which claims priority to EP 17 209 699.2, filed Dec. 21, 2017, the entire disclosures of all of which are hereby incorporated herein by reference.

### BACKGROUND

This disclosure relates to the field of disease tracking and potentially even supporting the diagnostics process. Specifically, it relates to a method of assessing a muscular disability and, preferably, spinal muscular atrophy (SMA) in a subject comprising the steps of determining at least one performance parameter from a dataset of pressure force measurements of the individual finger strength from said subject using a mobile device, and comparing the determined at least one performance parameter to a reference, whereby the muscular disability and, preferably, SMA will be assessed. This disclosure also relates to a mobile device comprising a processor, at least one pressure sensor and a database as well as software which is tangibly embedded to said device and, when running on said device, carries out the method of this disclosure as well as the use of such a device for assessing a muscular disability and, preferably, SMA.

Spinal muscular atrophy (SMA) is an autosomal recessive disease also called proximal spinal muscular atrophy and 5q spinal muscular atrophy. It is a life-threatening, neuromuscular disorder with low prevalence associated with loss of motor neurons and progressive muscle wasting.

The disorder is caused by a genetic defect in the SMN1 gene (Brzustowicz, 1990, Lefebvre 1995). This gene encodes the SMN protein which is wide-spread expressed in all eukaryotic cells and necessary for survival of motor neurons. Reduced levels of the protein result in loss of function of neuronal cells in the anterior horn of the spinal cord. As a consequence of the loss of neuronal function, atrophy of skeletal muscles occurs.

Spinal muscular atrophy manifests in various degrees of severity, which all have in common progressive muscle wasting and mobility impairment. Proximal muscles and respiratory muscles are affected first. Other body systems may be affected as well, particularly in early-onset forms of the disorder. SMA is the most common genetic cause of infant death.

Four different types of SMA are described. Four different types of SMA are known. The infantile SMA or SMA1 (Werdnig-Hoffmann disease) is a severe form that manifests in the first months of life, usually with a quick and unexpected onset (“floppy baby syndrome”). The intermediate SMA or SMA2 (Dubowitz disease) affects children who are never able to stand and walk but who are able to maintain a sitting position at least some time in their life. The juvenile SMA or SMA3 (Kugelberg-Welander disease) manifests, typically, after 12 months of age and describes people with SMA3 who are able to walk without support at some time, although many later lose this ability. The adult SMA or SMA4 manifests, usually, after the third decade of life with gradual weakening of muscles that affects proximal muscles of the extremities frequently requiring the person to use a wheelchair for mobility.

For all SMA types, typical symptoms are hypotonia associated with absent reflexes, fibrillation in the electromyogram as well as muscle denervation and (sometimes) serum creatine kinase increase (Rutkove 2010).

While the above symptoms suggest SMA, the diagnosis can only be confirmed with certainty through genetic testing for bi-allelic deletion of exon 7 of the SMN1 gene. Genetic testing is usually carried out using a blood sample, and MLPA is one of more frequently used gene sequencing techniques, as it also allows establishing the number of SMN2 gene copies.

Preimplantation or prenatal genetic testing is also available for SMA. In particular, preimplantation genetic diagnosis can be used to screen for SMA-affected embryos during in-vitro fertilization. Prenatal testing for SMA is possible through chorionic villus sampling, cell-free fetal DNA analysis and other methods. However, theses genetic testing methods are only suitable if there is already suspicion for the potential development of SMA, e.g., due to the parents medical history.

Thus, Nusinersen (Spinraza™) is the only approved drug for the treatment of SMA. It is a modified antisense oligonucleotide which targets the intronic splicer N1. In addition to drug treatment, patients suffering from SMA typically require special medical care, in particular with respect to orthopaedics, mobility support, respiratory care, nutrition, cardiology and mental health.

Since SMA is a clinically heterogeneous disease of the CNS, diagnostic tools are needed that allow a reliable diagnosis and identification of the present disease status and symptom progression and can, thus, aid an accurate treatment.

### SUMMARY

This disclosure teaches means and methods complying with the aforementioned needs. The technical problem is solved by the embodiments characterized in the claims and described herein below. Thus, this disclosure relates to a method assessing a muscular disability and, preferably, spinal muscular atrophy (SMA) in a subject comprising the steps of:
a) determining at least one performance parameter from a dataset of pressure measurements of the individual finger strength from said subject using a mobile device; and
b) comparing the determined at least one performance parameter to a reference, whereby the muscular disability and, preferably, SMA will be assessed.

Typically, the method further comprises the step of (c) diagnosing the muscular disability and, preferably, SMA in a subject based on the comparison carried out in step (b).

In some embodiments, the method may also comprise prior to step (a) the step of obtaining from the subject using a mobile device a dataset of pressure measurements during predetermined activity performed by the subject. However, typically the method is an ex vivo method carried out on an existing dataset of activity measurements of a subject which does not require any physical interaction with the said subject.

The method as referred to in accordance with this disclosure includes a method which essentially consists of the aforementioned steps or a method which may include additional steps.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “performance parameter,” “sensor,” and “pressure sensor,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “particularly,” “more particularly,” “specifically,” “more specifically,” “typically,” and “more typically” or similar terms are used in conjunction with additional/alternative features, without restricting alternative possibilities. Thus, features introduced by these terms are additional/alternative features and are not intended to restrict the scope of the claims in any way. The invention may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of this disclosure.

The method may be carried out on the mobile device by the subject once the dataset of pressure measurements has been acquired. Thus, the mobile device and the device acquiring the dataset may be physically identical, i.e., the same device. Such a mobile device shall have a data acquisition unit which typically comprises means for data acquisition, i.e., means which detect or measure either quantitatively or qualitatively physical and/or chemical parameters and transform them into electronic signals transmitted to the evaluation unit in the mobile device used for carrying out the method according to this disclosure. The data acquisition unit comprises means for data acquisition, i.e., means which detect or measure either quantitatively or qualitatively physical and/or chemical parameters and transform them into electronic signals transmitted to the device being remote from the mobile device and used for carrying out the method according to this disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors and the like. The evaluation unit typically comprises a processor and a database as well as software which is tangibly embedded to said device and, when running on said device, carries out the method of this disclosure. More typically, such a mobile device may also comprise a user interface, such as a screen, which allows for providing the result of the analysis carried out by the evaluation unit to a user.

Alternatively, it may be carried out on a device being remote with respect to the mobile device that has been used to acquire the said dataset. In this case, the mobile device shall merely comprise means for data acquisition, i.e., means which detect or measure either quantitatively or qualitatively physical and/or chemical parameters and transform them into electronic signals transmitted to the device being remote from the mobile device and used for carrying out the method according to this disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, humidity sensors, pedometer, heart rate detectors, fingerprint detectors, touch sensors, voice recorders, light sensors, pressure sensors, location data detectors, cameras, sweat analysis sensors, GPS, Balistocardiography, and the like. Thus, the mobile device and the device used for carrying out the method of this disclosure may be physically different devices. In this case, the mobile device may correspond with the device used for carrying out the method of this disclosure by any means for data transmission. Such data transmission may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Accordingly, for carrying out the method of this disclosure, the only requirement is the presence of a dataset of pressure measurements obtained from a subject using a mobile device. The said dataset may also be transmitted or stored from the acquiring mobile device on a permanent or temporary memory device which subsequently can be used to transfer the data to the device used for carrying out the method of this disclosure. The remote device which carries out the method of this disclosure in this setup typically comprises a processor and a database as well as software which is tangibly embedded to said device and, when running on said device, carries out the method of this disclosure. More typically, the said device may also comprise a user interface, such as a screen, which allows for providing the result of the analysis carried out by the evaluation unit to a user.

The term “assessing” as used herein refers to determining or providing an aid for diagnosing whether a subject suffers from a muscular disability and, preferably, SMA, or not. As will be understood by those skilled in the art, such an assessment, although preferred to be, may usually not be correct for 100% of the investigated subjects. The term, however, requires that a statistically significant portion of subjects can be correctly assessed and, thus, identified as suffering from a muscular disability or SMA. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05. Thus, the method of this disclosure, typically, aids the identification of a muscular disability or SMA by providing a means for evaluating a dataset of pressure measurements. The term also encompasses any kind of diagnosing, monitoring or staging of SMA and, in particular, relates to assessing, diagnosing, monitoring and/or staging of any symptom or progression of any symptom associated with a muscular disability and, preferably, SMA.

A “muscular disability” as referred to herein is a condition which is accompanied by a disabled muscle function. Typically, such a muscular disability may be caused by a disease or disorder such as muscular atrophy and, more typically, it may be a neuromuscular disease such as spinal muscular atrophy. The term “spinal muscular atrophy (SMA)” as used herein relates to a neuromuscular disease which is characterized by the loss of motor neuron function, typically, in the spinal chord. As a consequence of the loss of motor neuron function, typically, muscle atrophy occurs resulting in an early dead of the affected subjects. The disease is caused by an inherited genetic defect in the SMN1 gene. The SMN protein encoded by said gene is required for motor neuron survival. The disease is inherited in an autosomal recessive manner.

Symptoms associated with SMA include areflexia, in particular, of the extremities, muscle weakness and poor muscle tone, difficulties in completing developmental phases in childhood, as a consequence of weakness of respiratory muscles, breathing problems occurs as well as secretion accumulation in the lung, as well as difficulties in sucking, swallowing and feeding/eating. Four different types of SMA are known.

The infantile SMA or SMA1 (Werdnig-Hoffmann disease) is a severe form that manifests in the first months of life, usually with a quick and unexpected onset (“floppy baby syndrome”). A rapid motor neuron death causes inefficiency of the major body organs, in particular, of the respiratory system, and pneumonia-induced respiratory failure is the most frequent cause of death. Unless placed on mechanical ventilation, babies diagnosed with SMA1 do not generally live past two years of age, with death occurring as early as within weeks in the most severe cases, sometimes termed SMA0. With proper respiratory support, those with milder SMA1 phenotypes accounting for around 10% of SMA1 cases are known to live into adolescence and adulthood.

The intermediate SMA or SMA2 (Dubowitz disease) affects children who are never able to stand and walk but who are able to maintain a sitting position at least some time in their life. The onset of weakness is usually noticed some time between 6 and 18 months. The progress is known to vary. Some people gradually grow weaker over time while others through careful maintenance avoid any progression. Scoliosis may be present in these children, and correction with a brace may help improve respiration. Muscles are weakened, and the respiratory system is a major concern. Life expectancy is somewhat reduced but most people with SMA2 live well into adulthood.

The juvenile SMA or SMA3 (Kugelberg-Welander disease) manifests, typically, after 12 months of age and describes people with SMA3 who are able to walk without support at some time, although many later lose this ability. Respiratory involvement is less noticeable, and life expectancy is normal or near normal.

The adult SMA or SMA4 manifests, usually, after the third decade of life with gradual weakening of muscles that affects proximal muscles of the extremities frequently requiring the person to use a wheelchair for mobility. Other complications are rare, and life expectancy is unaffected.

Typically, SMA in accordance with this disclosure is SMA1 (Werdnig-Hoffmann disease), SMA2 (Dubowitz disease), SMA3 (Kugelberg-Welander diseases) or SMA4.

SMA is typically diagnosed by the presence of the hypotonia and the absence of reflexes. Both can be measured by standard techniques by the clinician in a hospital including electromyography. Sometimes, serum creatine kinase may be increased as a biochemical parameter. Moreover, genetic testing is also possible, in particular, as prenatal diagnostics or carrier screening.

The term “subject” as used herein relates to animals and, typically, to mammals. In particular, the subject is a primate and, most typically, a human. The subject in accordance with this disclosure shall suffer from or shall be suspected to suffer from a muscular disability and, preferably, SMA, i.e., it may already show some or all of the symptoms associated with the said disease.

The term “at least one” means that one or more performance parameters may be determined in accordance with this disclosure, i.e., at least two, at least three, at least four, at least five, at least six, at least seven, at least eight, at least nine or at least ten or even more different performance parameters. Thus, there is no upper limit for the number of different performance parameters which can be determined in accordance with the method of this disclosure. Typically, however, there will be between one and four different performance parameters per dataset of pressure measurement determined. More typically, the parameter(s) are selected from the group consisting of: peak pressure, integral pressure, pressure profile over time, and oscillations of pressure.

The term “performance parameter” as used herein refers to a parameter which is indicative for the capability of a subject to exert finger pressure. More typically, the performance parameter is selected from the group consisting of: peak pressure, integral pressure, pressure profile over time, and oscillations of pressure. Depending on the type of activity which is measured, the performance parameter can be derived from the dataset acquired by the pressure measurement performed on the subject. Particular performance parameters to be used in accordance with this disclosure are listed elsewhere herein in more detail.

The term “dataset of pressure measurements” refers to the entirety of data which has been acquired by the mobile device from a subject during pressure measurements or any subset of said data useful for deriving the performance parameter.

The term “individual finger strength” as used herein refers to force levels which can be exerted by a finger. This includes the capability of applying a pressure peak, the capability of applying a certain pressure level over time (integral pressure) and/or the capability of maintaining a pressure over time.

In the following, particular envisaged pressure tests and means for measuring by a mobile device in accordance with the method of this disclosure are specified.

In an embodiment, the mobile device is, thus, adapted for performing or acquiring data from a pressure test (so-called “ring-a-bell test”) in which the maximum pressure which can be exerted by a finger of a subject is measured. Moreover, the test is, typically, also configured to measure the duration of maximum pressure application. The dataset acquired from such test allows identifying the peak pressure, the integral pressure as well as the pressure profile over time. The test may require calibration with respect to the maximum force which can be applied by a finger of the subject first. Moreover, there are sensor specific limitations which shall be regarded. In order to measure pressure in a range which is below the sensor intrinsic saturation, the test may be configured to avoid application of maximum pressure. This can be advantageously achieved by tests such as the carry-the-egg test described elsewhere herein in detail.

The aforementioned pressure measurements can be made by a mobile device such as a smart phone by using the Force Touch technology or 3D Touch technology. Force Touch technology uses electrodes for sensing force which line the edges of a screen of the mobile device. Said electrodes determine the pressure applied to the screen. Accordingly, a test may display certain tasks on the screen which require pressing said screen with the finger thereby applying force in certain strength or over a certain time. The measured parameters from the electrodes are subsequently relayed to an electromagnetic linear actuator that oscillates back and forth. Said actuator produces data for a dataset of force measurements in accordance with this disclosure. 3D Touch technology works by using capacitive sensors integrated directly into the screen. When a press is detected, these capacitive sensors measure microscopic changes in the distance between the backlight and the cover glass. These data are then combined with accelerometer data and touch sensors data to complete the data of the dataset of force measurements which can be used for determining at least one performance parameter by a suitable algorithm running on, e.g., an evaluation unit. Further details on a force touch sensor to be typically included in a mobile device used to generate the dataset of force measurements to be used in the method of the present is described in U.S. Pat. No. 8,633,916. 3D Touch technology force sensors to be typically included in a mobile device used to generate the dataset of force measurements to be used in the method of the present is described in WO2015/106183. Further suitable force measurement sensors to be used in mobile devices are described in any one of EP 2 368 170, U.S. Pat. No. 9,116,569, EP 2 635 957, U.S. Pat. No. 8,952,987 or U.S. Publication No. 2015/0097791.

In another embodiment, the mobile device is adapted for performing or acquiring a data from a further pressure test (so-called “carry-the-egg test”) configured to measure the ability to sustain a controlled amount of pressure via a finger over a defined period of time. The dataset acquired from such test allow identifying the oscillation of pressure and a pressure profile over time. The test may require calibration with respect to a comfort pressure level, i.e., thresholds for the comfort level of pressure may need to be identified first. Moreover, the test shall be configured such that the measurement is carried out below the sensor intrinsic saturation for pressure measurements. The aforementioned pressure measurements can be made by a mobile device such as a smart phone by using the force touch technology or 3D Touch technology as defined elsewhere herein or analogue technology that allows measurement of force or pressure on a touch screen.

Both tests may be implemented on the mobile device by a computer program code which requests that the subject user performs certain tasks which allow for potential calibration and the actual pressure measurements. Typically, such tasks may be masked within an entertaining exercise or game which requires that the subject performs the tasks in a playfully and, thus, comfortable manner on the device. By using said game setup, the tasks can be, in particular, also be performed by children or subjects having impaired cognitive capabilities. Moreover, the gaming character of the test may also improve the overall motivation of the subjects to perform the tests. Typically envisaged examples for the pressure measurement tests are described in the accompanying Examples below in more detail.

It will be understood that the mobile device to be applied in accordance with this disclosure may be adapted to perform one or more of the aforementioned force measurement tests. In particular, it may be adapted to perform both tests.

Depending on the mobile device, pressure measurements measuring peak pressure, the capability of applying a certain pressure level over time (integral pressure) and/or the capability of maintaining a pressure over time (pressure profile) can also be performed during other uses of the mobile device where actions are performed which allow for the said pressure measurements (passive tests) to be recorded without the user focusing on it. Typically, if a smart phone is used as a mobile device, the subject (user) will usually perform a variety of touch controlled tasks which involve finger pressure-driven interactions with the screen. Typically, tapping will occur when telephone numbers are dialed or other standard activities are performed, e.g., internet queries are made or the like. The pressure applied by the fingers during performing such tasks may be analyzed over a certain time for calibration purposes and for providing a reference. Typically, peak pressure measurements may be performed during, e.g., tapping tasks such as dialing or the applied pressure may be integrated over a certain time window to yield an integral pressure. Change in the peak force, the integral pressure or a task specific pressure profile with respect to the reference may subsequently be used in the method according to this disclosure to be applied for investigating the dataset obtained from said (passive) pressure measurements.

Moreover, tapping and other pressure applying activities shall occur during the performance of the further tests mentioned below. Pressure measurements may also be performed as passive tests during said further tests.

Moreover, the mobile device may be adapted to perform further tests which are relevant for assessing SMA or muscular disabilities. Accordingly, further data may be processed in the method of this disclosure as well. These further data are typically suitable for further strengthening the assessment of SMA or muscular disability in a subject. Particular envisaged tests which investigate distal motor function (i.e., tapping, drawing and pinching abilities of fingers), axial motor function (i.e., lifting, twisting, tightrope and water pouring abilities of the subject), and/or central motor function (i.e., voice abilities) described in more detail below. In addition, surveys on overall well-being and cognitive capabilities may be regarded as well.

Particular envisaged further tests to be implemented on the mobile device for acquiring data which may be typically included into the dataset to be investigated by the method of this disclosure are selected from the following tests:

(1) Tests for distal motor functions: Tapping test, draw a shape test, and squeeze a shape test

The mobile device may be further adapted for performing or acquiring a data from a further test for distal motor function (so-called “tapping test”) configured to measure dexterity and distal weakness of the fingers. The dataset acquired from such test allow identifying the finger speed, precision of finger movements and finger travel time and distance.

The mobile device may be further adapted for performing or acquiring a data from a further test for distal motor function (so-called “draw a shape test”) configured to measure dexterity and distal weakness of the fingers. The dataset acquired from such test allow identifying the precision of finger movements, pressure profile and speed profile.

The aim of the “Draw a Shape” test is to assess fine finger control and stroke sequencing. The test is considered to cover the following aspects of impaired hand motor function: tremor and spasticity and impaired hand-eye coordination. The patients are instructed to hold the mobile device in the untested hand and draw on a touchscreen of the mobile device 6 prewritten alternating shapes of increasing complexity (linear, rectangular, circular, sinusoidal, and spiral; vide infra) with the second finger of the tested hand “as fast and as accurately as possible” within a maximum time of for instance 30 seconds. To draw a shape successfully the patient's finger has to slide continuously on the touchscreen and connect indicated start and end points passing through all indicated check points and keeping within the boundaries of the writing path as much as possible. The patient has maximum two attempts to successfully complete each of the 6 shapes. Test will be alternatingly performed with right and left hand. User will be instructed on daily alternation. The two linear shapes have each a specific number “a” of checkpoints to connect, i.e., “a-1” segments. The square shape has a specific number “b” of checkpoints to connect, i.e., “b-1” segments. The circular shape has a specific number “c” of checkpoints to connect, i.e., “c-1” segments. The eight-shape has a specific number “d” of checkpoints to connect, i.e., “d-1” segments. The spiral shape has a specific number “e” of checkpoints to connect, i.e., “e-1” segments. Completing the 6 shapes then implies to draw successfully a total of “(2a+b+c+d+e-6)” segments.

Typical Draw a Shape test performance parameters of interest:

Based on shape complexity, the linear and square shapes can be associated with a weighting factor (Wf) of 1, circular and sinusoidal shapes a weighting factor of 2, and the spiral shape a weighting factor of 3. A shape which is successfully completed on the second attempt can be associated with a weighting factor of 0.5. These weighting factors are numerical examples which can be changed in the context of this disclosure.
1. Shape completion performance scores:
   a. Number of successfully completed shapes (0 to 6) (ΣSh) per test.
   b. Number of shapes successfully completed at first attempt (0 to 6) (ΣSh₁).
   c. Number of shapes successfully completed at second attempt (0 to 6) (ΣSh₂).
   d. Number of failed/uncompleted shapes on all attempts (0 to 12) (ΣF).
   e. Shape completion score reflecting the number of successfully completed shapes adjusted with weighting factors for different complexity levels for respective shapes (0 to 10) (Σ[Sh*Wf]).
   f. Shape completion score reflecting the number of successfully completed shapes adjusted with weighting factors for different complexity levels for respective shapes and accounting for success at first vs second attempts (0 to 10) (Σ[Sh₁*Wf]+Σ[Sh₂*Wf*0.5]).
   g. Shape completion scores as defined in #1e, and #1f may account for speed at test completion if being multiplied by 30/t, where t would represent the time in seconds to complete the test.
   h. Overall and first attempt completion rate for each 6 individual shapes based on multiple testing within a certain period of time: (ΣSh₁)/ (ΣSh₁+ΣSh₂+ΣF) and (ΣSh₁+ΣSh₂)/(ΣSh₁+ΣSh₂+ΣF).
2. Segment completion and celerity performance scores/measures: (analysis based on best of two attempts [highest number of completed segments] for each shape, if applicable)
   a. Number of successfully completed segments (0 to [2a+b+c+d+e-6]) (ΣSe) per test.
   b. Mean celerity ([C], segments/second) of successfully completed segments: C=ΣSe/t, where t would represent the time in seconds to complete the test (max 30 seconds).
   c. Segment completion score reflecting the number of successfully completed segments adjusted with weighting factors for different complexity levels for respective shapes (Σ[Se*Wf]).
   d. Speed-adjusted and weighted segment completion score (Σ[Se*Wf]*30/t), where t would represent the time in seconds to complete the test.
   e. Shape-specific number of successfully completed segments for linear and square shapes (ΣSe_{LS}).
   f. Shape-specific number of successfully completed segments for circular and sinusoidal shapes (ΣSe_{CS}).
   g. Shape-specific number of successfully completed segments for spiral shape (ΣSe_{S}).
   h. Shape-specific mean linear celerity for successfully completed segments performed in linear and square shape testing: C_{L}=ΣSe_{LS}/t, where t would represent the cumulative epoch time in seconds elapsed from starting to finishing points of the corresponding successfully completed segments within these specific shapes.
   i. Shape-specific mean circular celerity for successfully completed segments performed in circular and sinusoidal shape testing: C_{C}=ΣSe_{CS}/t, where t would represent the cumulative epoch time in seconds elapsed from starting to finishing points of the corresponding successfully completed segments within these specific shapes.
   j. Shape-specific mean spiral celerity for successfully completed segments performed in the spiral shape testing: C_{S}=ΣSe_{S}/t, where t would represent the cumulative epoch time in seconds elapsed from starting to finishing points of the corresponding successfully completed segments within this specific shape.
3. Drawing precision performance scores/measures: (analysis based on best of two attempts [highest number of completed segments] for each shape, if applicable)
   a. Deviation (Dev) calculated as the sum of overall area under the curve (AUC) measures of integrated surface deviations between the drawn trajectory and the target drawing path from starting to ending checkpoints that were reached for each specific shapes divided by the total cumulative length of the corresponding target path within these shapes (from starting to ending checkpoints that were reached).
   b. Linear deviation (Dev_{L}) calculated as Dev in # 3a but specifically from the linear and square shape testing results.
   c. Circular deviation (Dev_{C}) calculated as Dev in # 3a but specifically from the circular and sinusoidal shape testing results.
   d. Spiral deviation (Dev_{S}) calculated as Dev in # 3a but specifically from the spiral shape testing results.
   e. Shape-specific deviation (Dev₁₋₆) calculated as Dev in # 3a but from each of the 6 distinct shape testing results separately, only applicable for those shapes where at least 3 segments were successfully completed within the best attempt.
   f. Continuous variable analysis of any other methods of calculating shape-specific or shape-agnostic overall deviation from the target trajectory.
4. Pressure profile measurement:
   a. Exerted average pressure.
   b. Deviation (Dev) calculated as the standard deviation of pressure.

The mobile device may be further adapted for performing or acquiring data from a further test for distal motor function (so-called “squeeze a shape test”) configured to measure dexterity and distal weakness of the fingers. The dataset acquired from such test allow identifying the precision and speed of finger movements and related pressure profiles. The test may require calibration with respect to the movement precision ability of the subject first.

The aim of the Squeeze a Shape test is to assess fine distal motor manipulation (gripping and grasping) and control by evaluating accuracy of pinch closed finger movement. The test is considered to cover the following aspects of impaired hand motor function: impaired gripping/grasping function, muscle weakness, and impaired hand-eye coordination. The patients are instructed to hold the mobile device in the untested hand and by touching the screen with two fingers from the same hand (thumb+second or thumb+third finger preferred) to squeeze/pinch as many round shapes (i.e., tomatoes) as they can during 30 seconds. Impaired fine motor manipulation will affect the performance. Test will be alternatingly performed with right and left hand. User will be instructed on daily alternation.

Typical Squeeze a Shape test performance parameters of interest:
1. Number of squeezed shapes:
   a. Total number of tomato shapes squeezed in 30 seconds (ΣSh).
   b. Total number of tomatoes squeezed at first attempt (ΣSh₁) in 30 seconds (a first attempt is detected as the first double contact on screen following a successful squeezing if not the very first attempt of the test).
2. Pinching precision measures:
   a. Pinching success rate (P_{SR}) defined as ΣSh divided by the total number of pinching (ΣP) attempts (measured as the total number of separately detected double finger contacts on screen) within the total duration of the test.
   b. Double touching asynchrony (DTA) measured as the lag time between first and second fingers touch the screen for all double contacts detected.
   c. Pinching target precision (P_{TP}) measured as the distance from equidistant point between the starting touch points of the two fingers at double contact to the centre of the tomato shape, for all double contacts detected.
   d. Pinching finger movement asymmetry (P_{FMA}) measured as the ratio between respective distances slid by the two fingers (shortest/longest) from the double contact starting points until reaching pinch gap, for all double contacts successfully pinching.
   e. Pinching finger velocity (P_{FV}) measured as the speed (mm/sec) of each one and/or both fingers sliding on the screen from time of double contact until reaching pinch gap, for all double contacts successfully pinching.
   f. Pinching finger asynchrony (P_{FA}) measured as the ratio between velocities of respective individual fingers sliding on the screen (slowest/fastest) from the time of double contact until reaching pinch gap, for all double contacts successfully pinching.
   g. Continuous variable analysis of 2a to 2f over time as well as their analysis by epochs of variable duration (5-15 seconds).
   h. Continuous variable analysis of integrated measures of deviation from target drawn trajectory for all tested shapes (in particular the spiral and square).
3. Pressure profile measurement:
   a. Exerted average pressure.
   b. Deviation (Dev) calculated as the standard deviation of pressure.
      (2) Tests for measuring axial motor function: lifting test, twisting test, walk the rope test and collect coins test

The mobile device may be further adapted for performing or acquiring a data from a further test for axial motor function (so-called “lifting test”) configured to measure upper extremity mobility (by lifting the mobile device), weakness and fatigue, proximal hypotonia, joint contractures and tremor. The dataset acquired from such test allow identifying the precision and speed of upper extremity movements. The test may require calibration with respect to the movement precision ability of the subject first.

The mobile device may be further adapted for performing or acquiring a data from a further test for axial and proximal motor function motor function (so-called “twisting test”) configured to measure upper extremity mobility (e.g., by twisting the mobile device), weakness and fatigue, proximal hypotonia, joint contractures and tremor. For this test, the patient has to hold the phone in the palm of his/her hand and turn the phone screen up and down repeatedly.

The dataset acquired from such test allow identifying the precision and speed and number of twists (rotations of the wrist). The test may require calibration with respect to the movement precision ability of the subject first.

The mobile device may be further adapted for performing or acquiring a data from a further test for axial motor function (so-called “walk the rope test”) configured to measure proximal hypotonia in the upper extremities. The dataset acquired from such test allow identifying the number, size and velocity of correct movements. The test may require calibration with respect to the counterbalance and imbalance abilities of the subject first.

The mobile device may be further adapted for performing or acquiring data from a further test for axial motor function (so-called “collect coins test”) configured to measure upper extremity mobility (by moving the mobile device), weakness and fatigue. The dataset acquired from such test allow identifying the extend of the axial rotation movement, the speed and the number of movements over time as well as reaction times as response to the progressing game situation (i.e., the ball needs to be alternated by the user between opposing sites of the screen). The test may require calibration with respect to the movement precision ability of the subject first.

(3) Tests for central motor function: voice test

The mobile device may be further adapted for performing or acquiring a data from a further test for central motor function (so-called “voice test”) configured to measure proximal central motoric functions by measuring voicing capabilities.

Typically, the aforementioned tests may be implemented on the mobile device as well by a computer program code which requests that the subject user performs certain tasks which allow for calibration and the force measurements. Typically, such tasks may be masked within a game which requires that the subject performs the tasks in a playfully and, thus, comfortable and relaxed manner on the device. By using said game setup, the tasks can be, in particular, also be performed by children or subjects having impaired cognitive capabilities. Moreover, the gaming character of the test may also improve the overall motivation of the subjects to perform the tests. Typically envisaged examples for the aforementioned tests are described in the accompanying Examples below in more detail.

In yet an embodiment of the method of this disclosure, the mobile device from which the dataset is obtained is configured in addition to the dataset of pressure measurements to provide at least data from at least one of the tests for distal motor function, axial motor function and/or central motor function and, more typically, for any one of these types of data.

However, in accordance with the method of this disclosure, further clinical, biochemical or genetic parameters may be considered. Typically, said further parameters may be obtained from electromyography, measurement of creatine kinase and/or genetic testing for, e.g., SMN1, SMN2 and/or VABP gene mutations and/or aberrations.

The term “mobile device” as used herein refers to any portable device which comprises at least a pressure sensor and data-recording equipment suitable for obtaining the dataset of pressure measurements. This may also require a data processor and storage unit as well as a display for electronically simulating a pressure measurement test on the mobile device. Moreover, from the activity of the subject data shall be recorded and compiled to a dataset which is to be evaluated by the method of this disclosure either on the mobile device itself or on a second device. Depending on the specific setup envisaged, it may be necessary that the mobile device comprises data transmission equipment in order to transfer the acquired dataset from the mobile device to further device. Particular well suited as mobile devices according to this disclosure are smartphones, portable multimedia devices or tablet computers. Alternatively, portable sensors with data recording and processing equipment may be used. Further, depending on the kind of activity test to be performed, the mobile device shall be adapted to display instructions for the subject regarding the activity to be carried out for the test. Particular envisaged activities to be carried out by the subject are described elsewhere herein and encompass the distal hypotonia tests as well as other tests described in this specification.

Determining at least one performance parameter can be achieved either by deriving a desired measured value from the dataset as the performance parameter directly. Alternatively, the performance parameter may integrate one or more measured values from the dataset and, thus, may be a derived from the dataset by mathematical operations such as calculations. Typically, the performance parameter is derived from the dataset by an automated algorithm, e.g., by a computer program which automatically derives the performance parameter from the dataset of activity measurements when tangibly embedded on a data processing device feed by the said dataset.

The term “reference” as used herein refers to a discriminator which allows assessing the muscular disability and, preferably, SMA in a subject. Such a discriminator may be a value for the performance parameter which is indicative for subjects suffering from the muscular disability and, preferably, SMA or subjects not suffering from the muscular disability and, preferably, SMA.

Such a value may be derived from one or more performance parameters of subjects known to suffer from the muscular disability and, preferably, SMA. Typically, the average or median may be used as a discriminator in such a case. If the determined performance parameter from the subject is identical to the reference or above a threshold derived from the reference, the subject can be identified as suffering from the muscular disability and, preferably, SMA in such a case. If the determined performance parameter differs from the reference and, in particular, is below the said threshold, the subject shall be identified as not suffering from the muscular disability and, preferably, SMA.

Similarly, a value may be derived from one or more performance parameters of subjects known not to suffer from the muscular disability and, preferably, SMA. Typically, the average or median may be used as a discriminator in such a case. If the determined performance parameter from the subject is identical to the reference or below a threshold derived from the reference, the subject can be identified as not suffering from the muscular disability and, preferably, SMA in such a case. If the determined performance parameter differs from the reference and, in particular, is above the said threshold, the subject shall be identified as suffering from the muscular disability and, preferably, SMA.

As an alternative, the reference may be a previously determined performance parameter from a dataset of pressure measurements which has been obtained from the same subject prior to the actual dataset. In such a case, a determined performance parameter determined from the actual dataset which differs with respect to the previously determined performance parameter shall be indicative for either an improvement or worsening depending on the previous status of the disease or a symptom accompanying it and the kind of activity represented by the performance parameter. The skilled person knows based on the kind of activity and previous performance parameter how the said parameter can be used as a reference.

Comparing the determined at least one performance parameter to a reference can be achieved by an automated comparison algorithm implemented on a data processing device such as a computer. Compared to each other are the values of a determined performance parameter and a reference for said determined performance parameter as specified elsewhere herein in detail. As a result of the comparison, it can be assessed whether the determined performance parameter is identical or differs from or is in a certain relation to the reference (e.g., is larger or lower than the reference). Based on said assessment, the subject can be identified as suffering from the muscular disability and, preferably, SMA (“rule-in”), or not (“rule-out”). For the assessment, the kind of reference will be taken into account as described elsewhere in connection with suitable references according to this disclosure.

Moreover, by determining the degree of difference between a determined performance parameter and a reference, a quantitative assessment of the muscular disability and, preferably, SMA in a subject shall be possible. It is to be understood that an improvement, worsening or unchanged overall disease condition or of symptoms thereof can be determined by comparing an actually determined performance parameter to an earlier determined one used as a reference. Based on quantitative differences in the value of the said performance parameter, the improvement, worsening or unchanged condition can be determined and, optionally, also quantified. If other references, such as references from subjects with SMA are used, it will be understood that the quantitative differences are meaningful if a certain disease stage can be allocated to the reference collective. Relative to this disease stage, worsening, improvement or unchanged disease condition can be determined in such a case and, optionally, also quantified.

The said diagnosis, i.e., the assessment of the muscular disability or SMA in the subject, is indicated to the subject or another person, such as a medical practitioner. Typically, this is achieved by displaying the diagnosis on a display of the mobile device or the evaluation device. Alternatively, a recommendation for a therapy, such as a drug treatment, or for a certain life style, e.g., a certain nutritional diet or rehabilitation measures, is provided automatically to the subject or other person. To this end, the established diagnosis is compared to recommendations allocated to different diagnosis in a database. Once the established diagnosis matches one of the stored and allocated diagnoses, a suitable recommendation can be identified due to the allocation of the recommendation to the stored diagnosis matching the established diagnosis. Accordingly, it is, typically, envisaged that the recommendations and diagnoses are present in form of a relational database. However, other arrangements which allow for the identification of suitable recommendations are also possible and known to the skilled artisan.

Moreover, the one or more performance parameter may also be stored on the mobile device or indicated to the subject, typically, in real-time. The stored performance parameters may be assembled into a time course or similar evaluation measures. Such evaluated performance parameters may be provided to the subject as a feedback for activity capabilities investigated in accordance with the method of this disclosure. Typically, such a feedback can be provided in electronic format on a suitable display of the mobile device and can be linked to a recommendation for a therapy as specified above or rehabilitation measures.

Further, the evaluated performance parameters may also be provided to medical practitioners in doctor's offices or hospitals as well as to other health care providers, such as, developers of diagnostic tests or drug developers in the context of clinical trials, health insurance providers or other stakeholders of the public or private health care system.

Typically, the method of this disclosure for assessing SMA in a subject may be carried out as follows:

First, at least one performance parameter is determined from an existing dataset of pressure measurements obtained from said subject using a mobile device. Said dataset may have been transmitted from the mobile device to an evaluating device, such as a computer, or may be processed in the mobile device in order to derive the at least one performance parameter from the dataset.

Second, the determined at least one performance parameter is compared to a reference by, e.g., using a computer-implemented comparison algorithm carried out by the data processor of the mobile device or by the evaluating device, e.g., the computer. The result of the comparison is assessed with respect to the reference used in the comparison and based on the said assessment the subject will be identified as a subject suffering from SMA, or not.

Third, the said diagnosis, i.e., the identification of the subject as being a subject suffering from SMA, or not, is indicated to the subject or other person, such as a medical practitioner. However, it will be understood that for a final clinical diagnosis or assessment further factors or parameters may be taken into account by the clinician.

The term “identification” as used herein refers to assessing whether a subject suffers from SMA with a certain likelihood. It will be understood that the assessment may, thus, not be correct for 100% of the cases. However, it is typically envisaged that a statistically significant portion of the investigated subjects can be assessed, i.e., identified as suffering from SMA. How statistical significance can be determined is described elsewhere herein. Identification as used herein refers, typically, to the provision of a hint rather to a final conclusion.

Alternatively, a recommendation for a therapy, such as a drug treatment, or for a certain life style, e.g., a certain nutritional diet, is provided automatically to the subject or another person. To this end, the established diagnosis is compared to recommendations allocated to different diagnosis in a database. Once the established diagnosis matches one of the stored and allocated diagnoses, a suitable recommendation can be identified due to the allocation of the recommendation to the stored diagnosis matching the established diagnosis. Typical recommendations involve therapy with Nusinersen, butyrates, valproic acid, hydroxyurea or riluzole.

Yet as an alternative or in addition, the at least one performance parameter underlying the diagnosis will be stored on the mobile device. Typically, it shall be evaluated together with other stored performance parameters by suitable evaluation tools, such as time course assembling algorithms, implemented on the mobile device which can assist electronically rehabilitation or therapy recommendation as specified elsewhere herein.

This disclosure, in light of the above, also specifically contemplates a method of assessing a muscular disability and, preferably, SMA in a subject comprising the steps of:
a) obtaining from said subject using a mobile device a dataset of pressure measurements during predetermined activity performed by the subject;
b) determining at least one performance parameter determined from a dataset of pressure measurements obtained from said subject using a mobile device;
c) comparing the determined at least one performance parameter to a reference; and
d) assessing the muscular disability and, preferably, SMA in a subject based on the comparison carried out in step (b), typically, by determining whether the subject suffers from the muscular disability and, preferably, SMA or not.

Advantageously, it has been found in the studies underlying this disclosure that performance parameters obtained from datasets of pressure measurements in SMA patients can be used as digital biomarkers for assessing SMA in those patients, i.e., identifying those patients which suffer from SMA. The said datasets can be acquired from the SMA patients in a convenient manner by using mobile devices such as the omnipresent smart phones, portable multimedia devices or tablet computers on which the subjects perform active or passive pressure tests. In particular, it was found in the studies underlying this disclosure that even datasets obtained by passive pressure measurements performed during other activities carried out on a smartphone are of sufficient quality for a meaningful assessment of SMA patients. The datasets acquired can be subsequently evaluated by the method of this disclosure for the performance parameter suitable as digital biomarker. Said evaluation can be carried out on the same mobile device or it can be carried out on a separate remote device. Moreover, by using such mobile devices, recommendations on life style or therapy can be provided to the patients directly, i.e., without the consultation of a medical practitioner in a doctor's office or hospital ambulance. Thanks to this disclosure, the life conditions of SMA patients can be adjusted more precisely to the actual disease status due to the use of actual determined performance parameters by the method of this disclosure. Thereby, drug treatments can be selected that are more efficient or dosage regimens can be adapted to the current status of the patient. It is to be understood that the method of this disclosure is, typically, a data evaluation method which requires an existing dataset of activity measurements from a subject. Within this dataset, the method determines at least one performance parameter which can be used for assessing SMA, i.e., which can be used as a digital biomarker for SMA. Moreover, it will be understood that the method of this disclosure using performance parameters from datasets of pressure measurements may also be applied for the assessment of muscular disabilities other than SMA. For such assessments the same principles shall apply as for SMA.

Accordingly, the method of this disclosure may be used for:
assessing the disease condition;
monitoring patients, in particular, in a real life, daily situation and on large scale;
supporting patients with life style and/or therapy recommendations;
investigating drug efficacy, e.g., also during clinical trials;
facilitating and/or aiding therapeutic decision making;
supporting hospital managements;
supporting rehabilitation measure management;
improving the disease condition as a rehabilitation instrument stimulating higher density cognitive, motoric and walking activity
supporting health insurances assessments and management; and/or
supporting decisions in public health management.

The explanations and definitions for the terms made above apply mutatis mutandis to the embodiments described herein below.

In the following, particular embodiments of the method of this disclosure are described:

In one embodiment, said SMA is SMA1 (Werdnig-Hoffmann disease), SMA2 (Dubowitz disease), SMA3 (Kugelberg-Welander diseases) or SMA4.

In another embodiment, the said at least one performance parameter is a parameter indicative for muscle hypotonia in an individual finger.

In yet an embodiment, the dataset of pressure measurements of the individual finger strength comprises data from the measurement the maximal pressure which can be exerted by a subject with an individual finger or for the capability of exerting pressure with an individual finger over time.

In an embodiment, said dataset further comprises data indicative for axial motor function and/or central motor function.

In an embodiment, said mobile device has been adapted for carrying out on the subject one or more of the pressure measurements referred to above. More typically, said mobile device is comprised in a smartphone, smartwatch, wearable sensor, portable multimedia device or tablet computer.

In an embodiment, said reference is at least one performance parameter derived from a dataset of pressure measurements of the individual finger strength from the said subject at a time point prior to the time point when the dataset of pressure measurements referred to in step a) has been obtained from the subject. More typically, a worsening between the determined at least one performance parameter and the reference is indicative for a subject with SMA.

In another embodiment, said reference is at least one performance parameter derived from a dataset of pressure measurements of the individual finger strength obtained from a subject or group of subjects known to suffer from SMA. More typically, a determined at least one performance parameter being essentially identical compared to the reference is indicative for a subject with SMA.

In yet another embodiment, said reference is at least one performance parameter derived from a dataset of pressure measurements of the individual finger strength obtained from a subject or group of subjects known not to suffer from SMA. More typically, a determined at least one performance parameter being worsened compared to the reference is indicative for a subject with SMA.

This disclosure also contemplates a computer program, computer program product or computer readable storage medium having tangibly embedded said computer program, wherein the computer program comprises instructions when run on a data processing device or computer carry out the method as specified above. Specifically, the present disclosure further encompasses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a dataset of pressure measurements obtained from the subject using a mobile, and
a data stream signal, typically encrypted, comprising the at least one performance parameter derived from the dataset of pressure measurements obtained from the subject using a mobile.

This disclosure further relates to a method for determining at least one performance parameter from a dataset of pressure measurements of the individual finger strength from said subject using a mobile device
a) deriving at least one performance parameter from a dataset of pressure measurements of the individual finger strength from said subject using a mobile device; and
b) comparing the determined at least one performance parameter to a reference, wherein, typically, said at least one performance parameter can aid assessing a muscular disability and, preferably, SMA in said subject.

This disclosure also encompasses a method for determining efficacy of a therapy against a muscular disability and, preferably, SMA comprising the steps of the method of this disclosure, in particular, the steps of a) determining at least one performance parameter from a dataset of pressure measurements of the individual finger strength from said subject using a mobile device, and b) comparing the determined at least one performance parameter to a reference, whereby the muscular disability and, preferably, SMA will be assessed or embodiments thereof specified elsewhere herein and the further step of determining a therapy response if improvement of a muscular disability and, preferably, SMA occurs in the subject upon therapy or determining a failure of response if worsening of the muscular disability and, preferably, SMA occurs in the subject upon therapy or if the muscular disability and, preferably, SMA remains unchanged.

The term “a therapy against a muscular disability and, preferably, SMA” as used herein refers to all kinds of medical treatments, including drug-based therapies, psychotherapy, physical-therapy and the like. The term also encompasses, life-style recommendations, rehabilitation measures, and recommendations of nutritional diets. Typically, the method encompasses recommendation of a drug-based therapy and, in particular, a therapy with a drug known to be useful for the treatment of muscular disability and, preferably, SMA. Such drug may be Nusinersen, butyrates, valproic acid, hydroxyurea or riluzole. Moreover, the aforementioned method may comprise in yet an embodiment the additional step of applying the recommended therapy to the subject.

Moreover, encompassed in accordance with this disclosure is a method for determining efficacy of a therapy against a muscular disability and, preferably, SMA comprising the steps of the aforementioned method of this disclosure (i.e., the method for assessing a muscular disability and, preferably, SMA in a subject) and the further step of determining a therapy response if improvement of a muscular disability and, preferably, SMA occurs in the subject upon therapy or determining a failure of response if worsening of the muscular disability and, preferably, SMA occurs in the subject upon therapy or if the muscular disability and, preferably, SMA remains unchanged.

The term “improvement” as referred to in accordance with this disclosure relates to any improvement of the overall disease condition or of individual symptoms thereof. Likewise, a “worsening” means any worsening of the overall disease condition or individual symptoms thereof. Since, e.g., SMA as a progressing disease is associated typically with a worsening of the overall disease condition and symptoms thereof, the worsening referred to in connection with the aforementioned method is an unexpected or untypical worsening which goes beyond the normal course of the disease. Unchanged SMA means that the overall disease condition and the symptoms accompanying it are within the normal course of the disease.

Moreover, this disclosure pertains to a method of monitoring a progressing muscular disability and, preferably, SMA in a subject comprising determining whether the muscular disability and, preferably, SMA improves, worsens or remains unchanged in a subject by carrying out the steps of the method of this disclosure, in particular, the steps of a) determining at least one performance parameter from a dataset of pressure measurements of the individual finger strength from said subject using a mobile device; and b) comparing the determined at least one performance parameter to a reference, whereby the muscular disability and, preferably, SMA will be assessed or embodiments thereof specified elsewhere herein at least two times during a predefined monitoring period.

The term “predefined monitoring period” as used herein refers to a predefined time period in which at least two times activity measurements are carried out. Typically, such a period may range from days to weeks to months to years depending on the disease progression to be expected for the individual subject. Within the monitoring period, the activity measurements and performance parameters are determined at a first time point which is usually the start of the monitoring period and at least one further time point. However, it is also possible that there are more than one further time point for activity measurements and performance parameter determination. In any event, the performance parameter(s) determined from the activity measurements of the first time point are compared to the performance parameters of subsequent time points. Based on such a comparison, quantitative differences can be identified which will be used to determine a worsening, improvement or unchanged disease condition during the predefined monitoring period.

This disclosure relates to a mobile device comprising a processor, at least one pressure sensor and a database as well as software being tangibly embedded into said device and, when running on said device, carries out the method of this disclosure.

The said mobile device is, thus, configured to be capable of acquiring the dataset and to determine the performance parameter therefrom. Moreover, it is configured to carry out the comparison to a reference and to establish the diagnosis, i.e., the identification of the subject as one suffering from a muscular disability and, preferably, SMA. Further details on how the mobile device can be designed for said purpose have been described elsewhere herein already in detail.

A system comprising a mobile device comprising at least one sensor and a remote device comprising a processor and a database as well as software which is tangibly embedded to said device and, when running on said device, carries out any of the methods of this disclosure, wherein said mobile device and said remote device are operatively linked to each other.

Under “operatively linked to each other” it is to be understood that the devices are connect as to allow data transfer from one device to the other device. Typically, it is envisaged that at least the mobile device which acquires data from the subject is connect to the remote device carrying out the steps of the methods of this disclosure such that the acquired data can be transmitted for processing to the remote device. However, the remote device may also transmit data to the mobile device such as signals controlling or supervising its proper function. The connection between the mobile device and the remote device may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Further details may be found elsewhere in this specification. For data acquisition, the mobile device may comprise a user interface such as screen or other equipment for data acquisition. Typically, the activity measurements can be performed on a screen comprised by a mobile device, wherein it will be understood that the said screen may have different sizes including, e.g., a 5.1 inch screen.

Moreover, it will be understood that this disclosure contemplates the use of the mobile device or the system according to this disclosure for assessing a muscular disability and, preferably, SMA on a dataset of pressure measurements of the individual finger strength from a subject.

This disclosure also contemplates the use of the mobile device or the system according to this disclosure for monitoring patients, in particular, in a real life, daily situation and on large scale.

Encompassed by this disclosure is furthermore the use of the mobile device or the system according to this disclosure for supporting patients with life style and/or therapy recommendations.

Yet, it will be understood that this disclosure contemplates the use of the mobile device or the system according to this disclosure for investigating drug safety and efficacy, e.g., also during clinical trials.

Further, this disclosure contemplates the use of the mobile device or the system according to this disclosure for facilitating and/or aiding therapeutic decision making.

Furthermore, this disclosure provides for the use of the mobile device or the system according to this disclosure for improving the disease condition as a rehabilitation instrument, and for supporting hospital management, rehabilitation measure management, health insurances assessments and management and/or supporting decisions in public health management.

In the following, further particular embodiments of this disclosure are listed:

Embodiment 1: A method assessing spinal muscular atrophy (SMA) in a subject comprising the steps of:
a) determining at least one performance parameter from a dataset of pressure measurements of the individual finger strength from said subject using a mobile device; and
b) comparing the determined at least one performance parameter to a reference, whereby SMA will be assessed.

Embodiment 2: The method of embodiment 1, wherein said SMA is SMA1 (Werdnig-Hoffmann disease), SMA2 (Dubowitz disease), SMA3 (Kugelberg-Welander diseases) or SMA4.

Embodiment 3: The method of embodiment 1 or 2, wherein the said at least one performance parameter is a parameter indicative for muscle hypotonia in an individual finger.

Embodiment 4: The method to any one of embodiments 1 to 3, wherein the dataset of pressure measurements of the individual finger strength comprises data from the measurement the maximal pressure which can be exerted by a subject with an individual finger or for the capability of exerting pressure with an individual finger over time.

Embodiment 5: The method of any one of embodiments 1 to 3, wherein said dataset further comprises data indicative for axial motor function and/or central motor function.

Embodiment 6: The method of any one of embodiments 1 to 5, wherein said mobile device has been adapted for carrying out on the subject one or more of the pressure measurements referred to in embodiment 4.

Embodiment 7: The method of embodiment 6, wherein said mobile device is comprised in a smartphone, smartwatch, wearable sensor, portable multimedia device or tablet computer.

Embodiment 8: The method of any one of embodiments 1 to 7, wherein said reference is at least one performance parameter derived from a dataset of pressure measurements of the individual finger strength from the said subject at a time point prior to the time point when the dataset of pressure measurements referred to in step a) has been obtained from the subject.

Embodiment 9: The method of embodiment 8, wherein a worsening between the determined at least one performance parameter and the reference is indicative for a subject with SMA.

Embodiment 10: The method of any one of embodiments 1 to 7, wherein said reference is at least one performance parameter derived from a dataset of pressure measurements of the individual finger strength obtained from a subject or group of subjects known to suffer from SMA.

Embodiment 11: The method of embodiment 10, wherein a determined at least one performance parameter being essentially identical compared to the reference is indicative for a subject with SMA.

Embodiment 12: The method of any one of embodiments 1 to 7, wherein said reference is at least one performance parameter derived from a dataset of pressure measurements of the individual finger strength obtained from a subject or group of subjects known not to suffer from SMA.

Embodiment 13: The method of embodiment 12, wherein a determined at least one performance parameter being worsened compared to the reference is indicative for a subject with SMA.

Embodiment 14: A mobile device comprising a processor, at least one pressure sensor and a database as well as software which is tangibly embedded to said device and, when running on said device, carries out the method of any one of embodiments 1 to 13.

Embodiment 15: A system comprising a mobile device comprising at least one pressure sensor and a remote device comprising a processor and a database as well as software which is tangibly embedded to said device and, when running on said device, carries out the method of any one of embodiments 1 to 13, wherein said mobile device and said remote device are operatively linked to each other.

Embodiment 16: Use of the mobile device according to embodiment 14 or the system of embodiment 15 for assessing SMA on a dataset of pressure measurements of the individual finger strength from a subject.

All references cited throughout this specification are herewith incorporated by reference with respect to their entire disclosure content and with respect to the specific disclosure contents mentioned in the specification.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIGS. 1A-1B show the results from a computer-implemented ring-a-bell test. The percentage of maximum pressure was correlated to the results of the daily activity (DA) score of the patients (FIG. 1A). Moreover, patients with high DA scores also showed strong results in the ring-a-bell test, while those with low DA scores showed only weak ring-a-bell test results (FIG. 1B).

FIGS. 2A-2B show the results from a computer-implemented carry-the-egg test. The percentage of maximum touch pressure required for performing the task was correlated to the results of the daily activity (DA) score of the patients (FIG. 2A). Moreover, patients with high DA scores also showed strong results in the carry-the-egg test, while those with low DA scores showed only weak carry-the-egg results (FIG. 2B).

FIGS. 3A-3B show the results of pressure measurements from a computer-implemented squeeze-a-shape (pinching) test. The measured finger pressure is correlated to the DA score (FIG. 3A). Moreover, patients with high DA scores also showed strong results in the squeeze-a-shape test, while those with low DA scores showed only weak results (FIG. 3B).

FIGS. 4A-4B show the results of pressure measurements from a computer-implemented draw-a-shape test. The measured drawing pressure is correlated to the DA score (FIG. 4A). Moreover, patients with high DA scores also showed strong results in the draw-a-shape test, while those with low DA scores showed only weak results (FIG. 4B).

FIGS. 5A-5D show computer-implemented versions of the ring-a-bell test (FIG. 5A), the carry-the-egg test (FIG. 5B), the squeeze-a-shape test (FIG. 5C), and the draw-a-shape test (FIG. 5D).

### DESCRIPTION AND EXAMPLES

The embodiments and examples described below are not intended to be exhaustive or to limit the invention to the precise forms disclosed in the following detailed description. Rather, the embodiments are chosen and described so that others skilled in the art may appreciate and understand the principles and practices of this disclosure. The following Examples merely illustrate the invention. They shall not be construed in a way as to limit the scope of the invention.

### Example 1

### Pressure Dataset Acquisition Using a Computer Implemented Test for Determining Finger Strength (Ring-a-Bell Test)

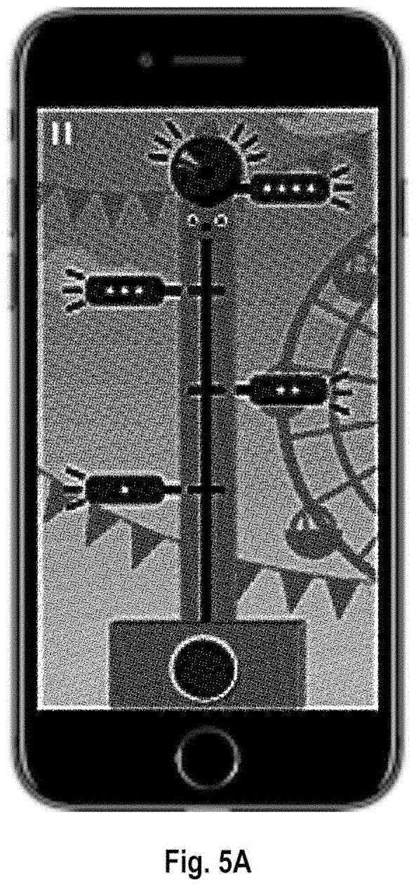

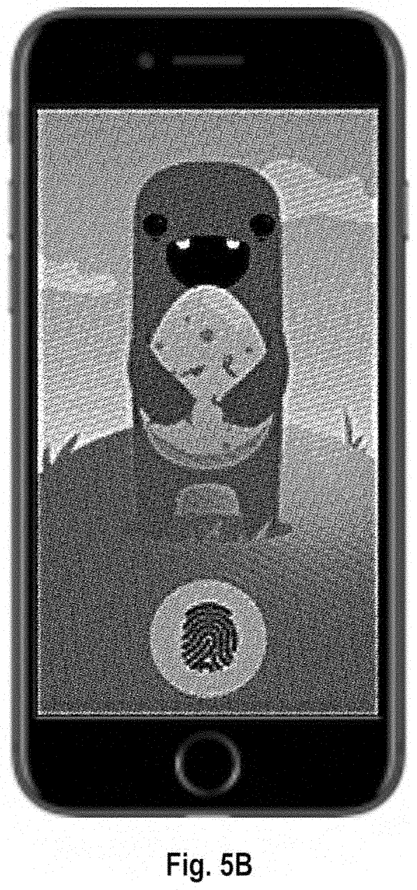

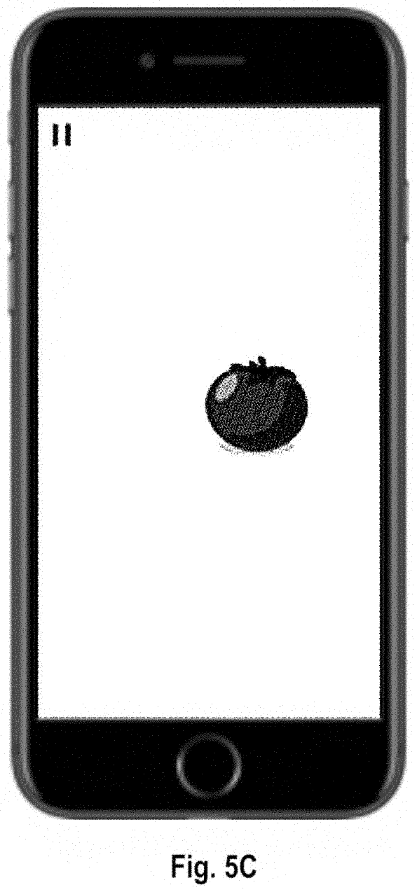

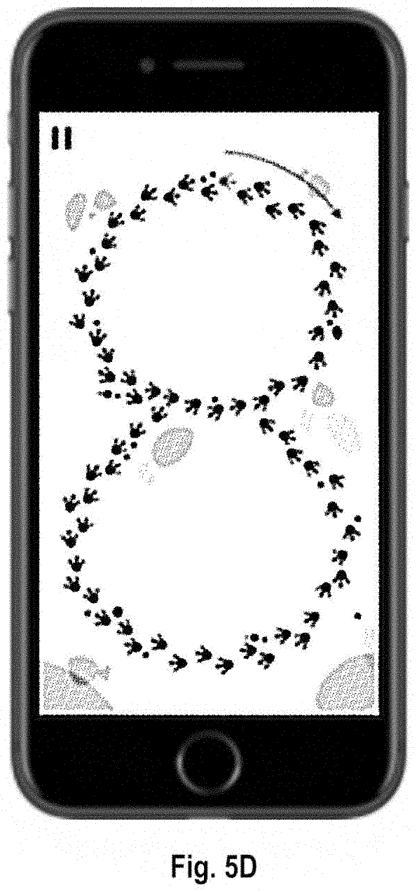

A test for measuring pressure exerted by a finger was implemented on a mobile phone (iPhone); see FIG. 5A. The patients shall exert maximum pressure on the surface of the display such that the bell will ring. The test was adapted to measure pressure application by a finger of a patient. The patient needs to play a game aiming to obtain maximum pressure and the duration of maximum pressure application (“ring-a-bell” test). The test required calibration with respect to the maximum pressure which can be applied by a finger of the subject first. The results of the ring-a-bell test are expressed as a percentage of said maximum pressure.

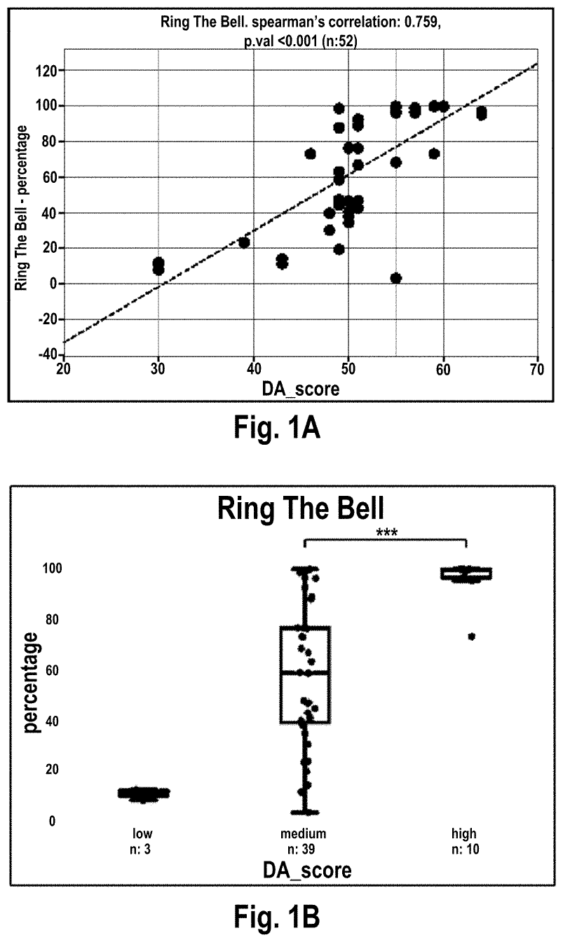

FIG. 1 shows the correlation of the daily activity of a patient and the results from the ring-a-bell test. It is apparent that patients with high daily activity show good results in the ring-a-bell test while those with low daily activity, i.e., those which are usually hardly affected by SMA, show weak results in the ring-a-bell test. 8 out of 23 tested patients showed ceiling effects.

### Example 2

### Pressure Dataset Acquisition Using a Computer Implemented Test for Determining Finger Strength (Carry-the-Egg Test)

Another test for measuring pressure exerted by a finger was implemented on a mobile phone (iPhone), the so-called “carry-the-egg test”; see FIG. 5B. Patients shall carry the schematic egg shown in the display. If too much pressure is applied, the carrying monster will destroy the egg, if too low pressure will be applied, it will drop the egg. The test was configured to measure the ability to sustain a controlled amount of pressure via a finger over a defined period of time. The dataset acquired from such test allows identifying the oscillation of and a pressure profile over time. The test may require calibration with respect to a pressure level required to perform the task. Moreover, the test was configured such that the measurement is carried out below the sensor intrinsic saturation for pressure measurements.

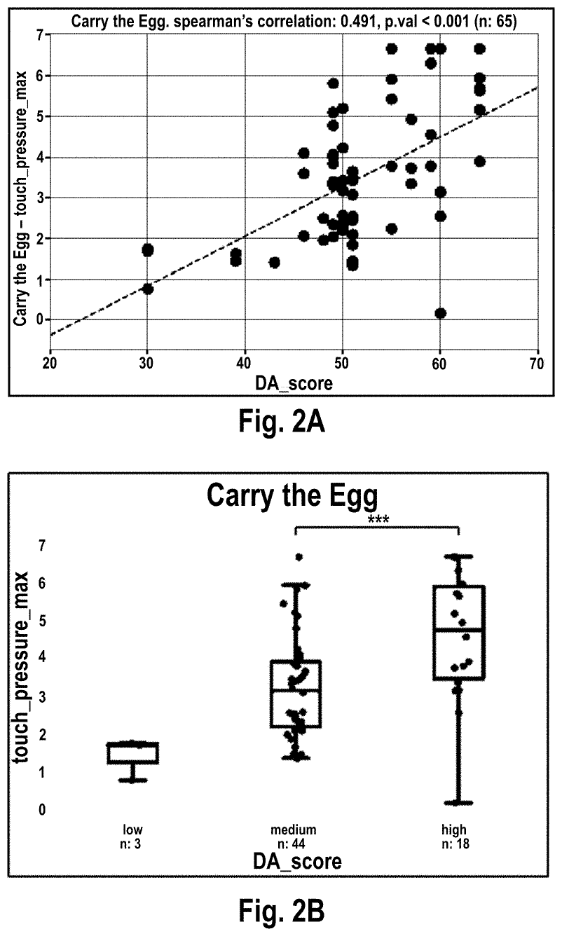

FIG. 2 shows the correlation of the daily activity of a patient and the results from the carry-the-egg test. It is apparent that patients with high daily activity show good results in the carry-the-egg test, while those with low daily activity, i.e., those which are usually hardly affected by SMA, show weak results in the carry-the-egg test.

### Example 3

### Pressure Dataset Acquisition Using Computer-Implemented Tests Pinching and Drawing

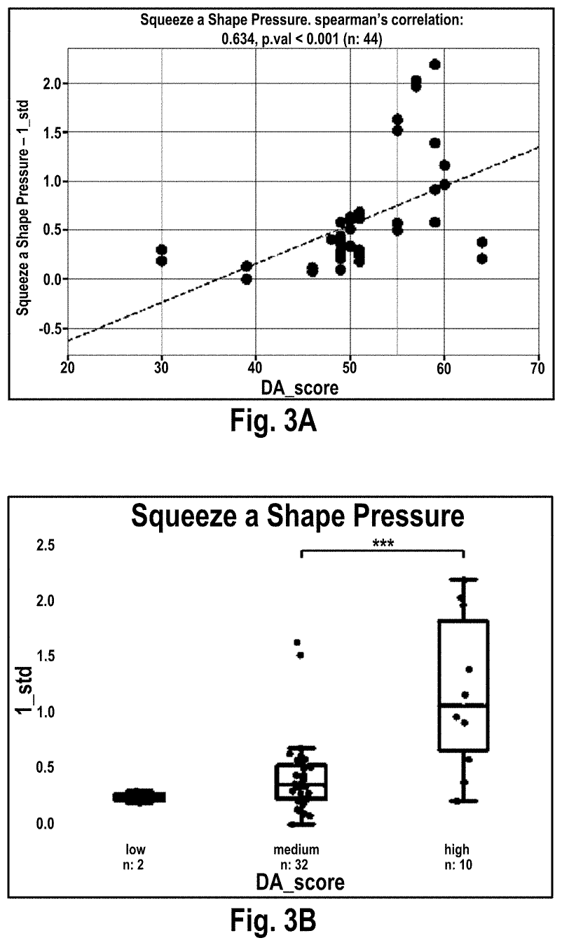

Another test for measuring pressure exerted by a finger was implemented on a mobile phone (iPhone), the so-called “pinching- or squeeze-a-shape test”; see FIG. 5C. The patients shall pinch or squeeze the shape indicated on the display, e.g., a schematic drawing of a tomato. It was configured to measure the pressure of a finger expressed as standard deviation of the shape pressure (pinching gesture) during a pinching movement of the surface of the display. FIG. 3 shows the correlation of the daily activity of a patient and the results from the squeeze-a-shape test. It is apparent that patients with high daily activity show good results in said test, while those with low daily activity, i.e., those which are usually hardly affected by SMA, show weak results.

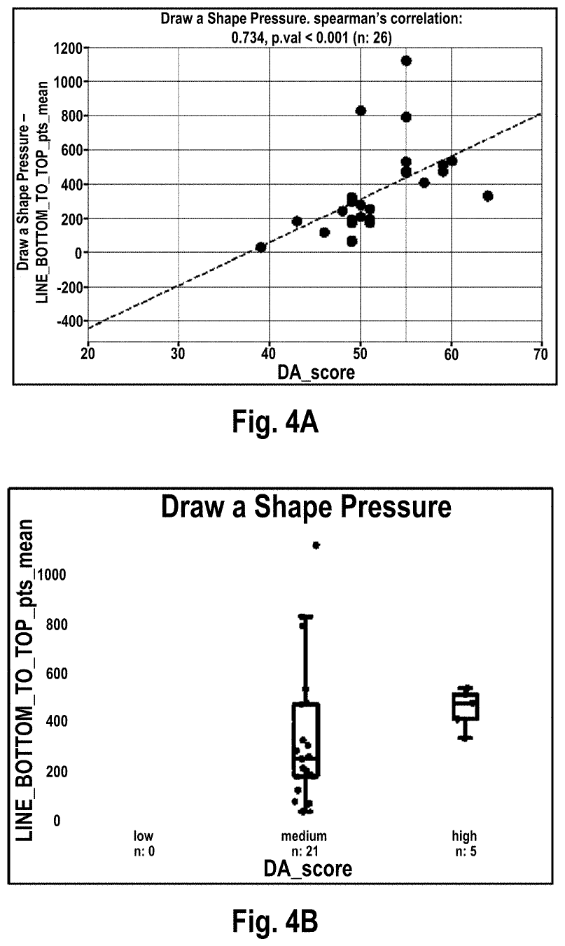

Similar results were obtained in a computer-implemented “draw-a-shape test”; see FIG. 5D and FIG. 4. The patient shall draw the shape depicted on the display. This test was configured to measure the momentum of the drawing, the drawing pressure and speed. The test result shows a differentiation of the patients regarding their capabilities for medium and strong patients. Weak patients could not perform the drawing test for all shapes.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method of assessing spinal muscular atrophy (SMA) in a subject, comprising:
a) determining at least one performance parameter from a dataset of pressure measurements of individual finger strength from said subject using a mobile device; and
b) comparing the determined at least one performance parameter to a reference; and
c) assessing SMA.

2. The method of claim 1, wherein said SMA is SMA1 (Werdnig-Hoffmann disease), SMA2 (Dubowitz disease), SMA3 (Kugelberg-Welander diseases) or SMA4.

3. The method of claim 1, wherein the at least one performance parameter is a parameter indicative for muscle hypotonia in an individual finger.

4. The method of claim 1, wherein the dataset of pressure measurements of the individual finger strength include data from the measurement the maximal pressure which can be exerted by a subject with an individual finger or for the capability of exerting pressure with an individual finger over time.

5. The method of claim 1, wherein the dataset includes data indicative of axial motor function and/or central motor function.

6. The method of claim 1, wherein the mobile device is configured to carry out on the subject one or more force measurements.

7. The method of claim 6, wherein the mobile device is a smartphone, smartwatch, wearable sensor, portable multimedia device or tablet computer.

8. The method of claim 1, wherein the reference is at least one performance parameter derived from a dataset of pressure measurements of the individual finger strength from the subject at a time point prior to the time point when the dataset of pressure measurements referred to in step a) has been obtained from the subject.

9. The method of claim 8, wherein a worsening between the determined at least one performance parameter and the reference is indicative for a subject with SMA.

10. The method of claim 1, wherein the reference is at least one performance parameter derived from a dataset of pressure measurements of the individual finger strength obtained from a subject or group of subjects known to suffer from SMA.

11. The method of claim 10, wherein a determined performance parameter being essentially identical to the reference indicates a subject with SMA.

12. The method of claim 1, wherein the reference is at least one performance parameter derived from a dataset of pressure measurements of the individual finger strength obtained from a subject or group of subjects known not to suffer from SMA.

13. The method of claim 12, wherein a determined at least one performance parameter being worse compared to the reference indicates a subject with SMA.

14. A mobile device comprising a processor, at least one pressure sensor, a database and a non-transitory computer-readable medium having embodied thereon computer-executable instructions, which when executed cause the mobile device to perform the method according to claim 1.

15. A system, comprising:
a mobile device having at least one pressure sensor; and
a remote device operatively linked to the mobile device, the remote device having a processor, a database and a non-transitory computer-readable medium having embodied thereon computer-executable instructions which when executed cause the mobile device to perform the method according to claim 1.

16. A method of assessing spinal muscular atrophy (SMA) in a subject, the method comprising:
a) collecting with a mobile device pressure measurements corresponding to predetermined activity performed by the subject;
b) forming a dataset from the collected pressure measurements;
c) using the mobile device to determine from the dataset a performance parameter of individual finger strength of the subject;
d) comparing the determined performance parameter to a reference; and
e) assessing SMA of the subject.

17. The method of claim 16, wherein the predetermined activity corresponds to finger pressure exerted by the subject on a touchscreen of the mobile device and read by a pressure sensor included in the mobile device.

18. The method of claim 16, wherein the mobile device is a smartphone, smartwatch, wearable sensor, portable multimedia device or tablet computer.

19. The method of claim 16, wherein the mobile device has a display configured to produce images that guide the subject in collecting the pressure measurements.

20. The method of claim 19, wherein the images are selected from the group consisting of: ring-a-bell, carry-the-egg, squeeze-a-shape and draw-a-shape.

21. The method of claim 16, wherein dataset includes data on the maximal pressure which can be exerted by a subject with an individual finger or for the capability of exerting pressure with an individual finger over time.
